SEQUENCE LISTING (11 sequences)

SEQ ID NO: 1 - DNA, 602 bp, Phyllosticta fallopiae
  source 1..602: mol_type = genomic DNA
gaaggatcattactgaaatgtaataacttctattgaaaggttccagagtaggcgctacaacgccgaaatgaccttctcacccttgtgtactcactatgttgctttggcgggtcgacctggttccgacccaggcggccggcgcccccagccttaactggccaggacgcccggctaagtgcccgccagtatacaaaactcaagaattcattttgtgaagtcctgatatatcatttaattgattaaaactttcaacaacggatctcttggttctggcatcgatgaagaacgcagcgaaatgcgataagtaatgtgaattgcagaattcagtgaatcatcgaatctttgaacgcacattgcgccctctggtattccggagggcatgcctgttcgagcgtcatttcaaccctcaagctctgcttggtattgggcaacgtccgctgccggacgggccttgaagacctcggcgacggcgtcctagcctcgagcgtagtagtaaaatatctcgctttggagtgctgggcgacggccgccggacaatcgaccttcggtctatttttccaaggttgacctcggatcaggtagggatacccgctgaacttaag

SEQ ID NO: 2 - DNA, 21 bp, synthetic construct
  source 1..21: mol_type = other DNA
tcctccgctttattgatatgc

SEQ ID NO: 3 - DNA, 21 bp, synthetic construct
  source 1..21: mol_type = other DNA
ggaagtaaagtcgtaacaagg

SEQ ID NO: 4 - DNA, 18 bp, synthetic construct
  source 1..18: mol_type = other DNA
aagggcgtatagttgagt

SEQ ID NO: 5 - DNA, 19 bp, synthetic construct
  source 1..19: mol_type = other DNA
aaatatctaggttgggtgt

SEQ ID NO: 6 - DNA, 18 bp, synthetic construct
  source 1..18: mol_type = other DNA
ggcttatcttatgtggca

SEQ ID NO: 7 - DNA, 18 bp, synthetic construct
  source 1..18: mol_type = other DNA
aaaggctgaaccgtatct

SEQ ID NO: 8 - DNA, 18 bp, synthetic construct
  source 1..18: mol_type = other DNA
cgcttggaagttggtagt

SEQ ID NO: 9 - DNA, 18 bp, synthetic construct
  source 1..18: mol_type = other DNA
gtgggtccctctgtattt

SEQ ID NO: 10 - DNA, 18 bp, synthetic construct
  source 1..18: mol_type = other DNA
tcatcaaggaagcaggag

SEQ ID NO: 11 - DNA, 18 bp, synthetic construct
  source 1..18: mol_type = other DNA
aaaggcgaaatcaaacag

(19) United States
(12) Patent Application Publication
(10) Pub. No.: US 2025/0230399 A1
(43) Pub. Date: Jul. 17, 2025

(54) ENDOPHYTIC FUNGUS OF DENDROBIUM NOBILE AND USE THEREOF

(71) Applicant: ZHEJIANG CHINESE MEDICAL UNIVERSITY, Hangzhou, Zhejiang (CN)

(72) Inventors: Luping QIN, Hangzhou (CN); Bo ZHU, Hangzhou (CN); Wenhua CHEN, Hangzhou (CN); Yujia LIU, Hangzhou (CN)

(21) Appl. No.: 19/096,768

(22) Filed: Apr. 1, 2025

Related U.S. Application Data
(63) Continuation of application No. PCT/CN2023/125380, filed on Oct. 19, 2023.

(30) Foreign Application Priority Data
Aug. 24, 2023 (CN) 202311074868.1

(51) Int. Cl. C12N 1/14; A01H 3/00; C12R 1/645

(52) U.S. Cl. CPC C12N 1/145; A01H 3/00; C12R 2001/645

(57) ABSTRACT

An endophytic fungus of Dendrobium nobile and a use thereof provided. The endophytic fungus is obtained by separating a living Orchidaceae perennial herb, Dendrobium nobile, by adopting an endophytic fungus separation and purification technology, with a category name of Phyllosticta fallopiae DNL19, which has been deposited in the China General Microbiological Culture Collection Center on Apr. 27, 2023, with a strain deposit number of CGMCC No. 40583. Meanwhile, a use of the endophytic fungus is provided, which promotes the growth of Dendrobium nobile, as well as the accumulation of an effective constituent dendrobine. The endophytic fungus brings broad application prospects in the field of yield and quality improvement of Dendrobium nobile medicinal materials.

### CROSS-REFERENCE TO RELATED APPLICATIONS

The present application is a National Stage of International Application No. PCT/CN2023/125380 filed on Oct. 19, 2023, which claims a priority to Chinese Patent Application No. 2023110748681, filed on Aug. 24, 2023, both of which are hereby incorporated by reference in their entireties.

### REFERENCE TO SEQUENCE LISTING

The present application is being filed along with a Sequence Listing in an electronic format. The Sequence Listing is provided as a file entitled with “DF244132US-Sequence Listing ST.26”, and created on Jan. 2, 2025, which is approximately 11.0 KB in size. The information in the electronic format of the Sequence Listing is incorporated herein by reference in its entirety.

### TECHNICAL FIELD

The present disclosure relates to the field of plant growth and secondary metabolism regulation, in particular to an endophytic fungus of Dendrobium nobile and a use thereof in promotion of growth and accumulation of dendrobine content in Dendrobium nobile Lindl.

### BACKGROUND

Dendrobium nobile Lindl., a Dendrobium perennial herb of family Orchidaceae, is mainly distributed in provinces such as Guizhou, Yunnan, Guangxi, Guangdong and Sichuan Provinces, China. According to the records in the Chinese Pharmacopoeia, Dendrobium nobile benefits the stomach and promotes fluids, and nourishes Yin and clears heat, and it is used for treating fever and dryness formed from fluid deficiency, dry mouth, irritability and thirst, deficiency of stomach Yin, dry vomiting due to insufficient food intake, persistent deficiency of heat after illness, Yin deficiency and excessive fire, and unclear vision. Modern medical research has shown that an active ingredient, dendrobine, of Dendrobium nobile has the effects of easing pains, resisting osteoporosis, resisting tumors, neuroprotection, etc.

The reproductive rate of Dendrobium nobile is low under natural conditions, wild resources are scarce, and the quality of artificial cultivation also varies. At present, the main ways to improve the quality of Dendrobium nobile and promote the accumulation of its active ingredients include breeding new varieties, improving cultivation methods, optimizing fertilization schemes, adding hormone inducers, etc. However, these methods have limitations of long cycles, insignificant effects, poor sustainability, and soil hardening.

Plant endophytic fungi are those who live in tissue and organs of healthy plants at certain or all stages and are commonly found in healthy plants. Research has found that the cross talk between endophytic fungi and their host plants is an important pathway for promoting plant growth and secondary metabolism. Endophytic fungi can promote the growth of the host plants and the accumulation of secondary metabolic components and deserved for further studies. For example, Yang He et al. used wild soybean endophytic fungi Y6R15 and Y2S2 solutions to treat soybeans and corn separately through “root irrigation” and “foliar spraying” methods and found changes in dry matter and root system indicators in seedling stages of corn and soybeans in different treatment groups, indicating that Y6R1 and Y2S2 had a growth promoting effect on corn and soybeans. Lan Taoju et al. screened 32 endophytic fungi isolated from stems of Bruguiera gymnorrhiza and obtained one dark septate endophyte (DSE) strain with a good growth promoting effect on Dendrobium officinale, designated as HS40. They found that after 6 months of inoculation of potted Dendrobium officinale seedlings with the strain HS40, their plant height, tiller number, total fresh weight, total dry weight, and stem polysaccharide content increased by 21.7%, 375.0%, 94.7%, 57.6% and 15.8%, respectively, when compared with a control group. Among them, the plant height, the total fresh weight, the total dry weight, and the stem polysaccharide content showed significant difference levels compared with the control group (P<0.01), as well as the tiller number (P<0.05). Ye Bingzhu et al. isolated 277 endophytic fungi from Anoectochilus roxburghii and Ludisia orchids, and found that two strains, J162 and J211, could be co-cultured with the medicinal plant Anoectochilus roxburghii in southern China, and could significantly enhance biosynthesis and accumulation of its active ingredients such as flavonoids, saponins, and polysaccharides.

However, there are few reports on the application research of using endophytic fungi to improve the yield and quality of Dendrobium nobile. Therefore, studying from the perspective of an ecosystem composed of Dendrobium nobile and its endophytic fungi is of great significance for improving the yield and quality of Dendrobium nobile and accelerating the industrial development of Dendrobium nobile.

### SUMMARY

Regarding the defects in the prior art, an objective of the present disclosure is to provide an endophytic fungus of Dendrobium nobile and a use thereof.

In the first aspect of the present disclosure, a plant endophytic fungus is provided. The endophytic fungus is obtained by separation and purification in leaves of an Orchidaceae plant, Dendrobium nobile, with a category name of Phyllosticta fallopiae DNL19. This strain has been deposited with a deposit number of CGMCC No. 40583 and a deposit date of Apr. 27, 2023, in the China General Microbiological Culture Collection Center (CGMCC) at No. 3, First Yard, Beichenxi Road, Chaoyang District, Beijing, 100101.

The endophytic fungus described in the present disclosure has solid culture characteristics that: culture is carried out in darkness at 25° C. on a potato-glucose-agar medium (PDA), the growth of the strain is slow, and fungal colonies are in dark grey, with white circular granular protrusions in the middle circle and smooth off-white fungal colonies in the outer circle, as shown in FIG. 1.

A formula of the PDA medium described in the present disclosure is: 200 g of potato, 20 g of glucose, 1000 mL of distilled water, and 15 g of agar, with a natural pH.

The endophytic fungus described in the present disclosure has morphological characteristics under an optical microscope that: the fungal colonies are in black, purple with short and thin hyphae and many branches, and internal diaphragms of part of the hyphae are seen indistinctly.

The endophytic fungus described in the present disclosure has morphological characteristics under a scanning microscope that: hyphae are circular as a whole, part of the hyphae are flat occasionally, walls are relatively smooth, and the hyphae are thin and have many branches, as shown in FIG. 2.

An ITS amplification base sequence of the endophytic fungus described in the present disclosure is as shown in SEQ ID NO. 1, and through sequence alignment for a sequencing result on the NCBI website (http://blast.ncbi.nlm.nih.gov/Blast.cgi), the homology with Phyllosticta fallopiae (NR147316) is 99.83%. The sequence alignment is performed with MEGA 7.0 software, a neighbor-joining (NJ) method is used for analysis, and one sequence is selected randomly and aligned repeatedly for 1000 times. An agarose gel electrophoresis diagram and a phylogenetic evolutionary tree of the endophytic fungus described in the present disclosure are as shown in FIG. 3 and FIG. 4.

In a second aspect of the present disclosure, a use of the above endophytic fungus is provided, including increases in fresh weight and dendrobine content of Dendrobium nobile.

In the above use, by means of co-culture of DNL19 fungus blocks and Dendrobium nobile plants, the increases in fresh weight and dendrobine content of Dendrobium nobile are promoted. As for the co-culture, it is required to inoculate the fungus blocks into ½ MS for co-culture with Dendrobium nobile tissue culture seedlings, and they are placed in a greenhouse with a temperature of 25±1° C., an illumination/dark cycle of 12 h/12 h and a relative humidity of 60% to 70%.

Further, it is mainly realized through the following technical solution: (1) taking the above endophytic fungus strain, under a sterile condition, picking a small amount of hyphae with an inoculating needle, inoculating the hyphae on a PDA medium (having a formula of 200 g of potato, 20 g of glucose, 15 g of agar, and 1000 mL of distilled water, with a natural pH), and carrying out dark culture for 7 d at 25° C. under a 65% humidity condition; and (2) under a sterile condition, punching fungus blocks at an edge of fungal colonies of the above strain with a 5 mm puncher to obtain the DNL19 fungus blocks, inoculating the fungus blocks in turn into the ½ MS medium for co-culture with the Dendrobium nobile tissue culture seedlings and placing the fungus blocks in the middle of the tissue culture seedlings. The inoculated Dendrobium nobile tissue culture seedlings are placed in the greenhouse with the temperature of 25±1° C., the illumination/dark cycle of 12 h/12 h and the relative humidity of 60% to 70% for performing co-culture (cultured for 60 d), and thus the fresh weight and dendrobine content of the Dendrobium nobile can be increased remarkably. A formula of the ½ MS medium (/L) is: 1.9 g of potassium nitrate, 1.65 g of ammonium nitrate, 0.17 g of potassium dihydrogen phosphate, 0.18 g of magnesium sulfate, 0.33 g of calcium chloride, 0.83 mg of potassium iodide, 6.2 mg of boric acid, 16.9 mg of manganese sulfate, 8.6 mg of zinc sulfate, 0.25 mg of sodium molybdate, 0.025 mg of copper sulfate, 0.1 g of inositol, 0.025 mg of cobalt chloride, 2.0 mg of glycine, 37.26 mg of EDTA sodium salt, 0.1 mg of vitamin B1, 15.2 mg of ferrous sulfate, 0.5 mg of nicotinic acid, 0.5 mg of vitamin B6, 15.0 g of sucrose, 7.0 g of agar powder, and 1000 mL of distilled water, with a pH of 5.8-6.0.

The present disclosure has the beneficial effects:

According to the endophytic fungus of Dendrobium nobile (Phyllosticta fallopiae DNL19) of the present disclosure, through co-culture with the Dendrobium nobile, the fresh weight and dendrobine content of the Dendrobium nobile can be increased remarkably. The endophytic fungus of the present disclosure has a remarkable improvement effect on the yield and quality of the Dendrobium nobile and brings broad application prospects for high-yield and high-quality cultivation of the Dendrobium nobile.

### BRIEF DESCRIPTION OF DRAWINGS

FIG. 1: a diagram of colonial morphology of the endophytic fungus Phyllosticta fallopiae DNL19 on a PDA medium.

FIG. 2: hypha morphology of the endophytic fungus Phyllosticta fallopiae DNL19 under an optical microscope and a scanning microscope.

FIG. 3: an ITS sequence gel electrophoresis diagram of Phyllosticta fallopiae DNL19 based on ITS5 and ITS4 primers amplification.

FIG. 4: a phylogenetic tree of Phyllosticta fallopiae DNL19 based on ITS4 and ITS5 primers amplification (NJ method).

FIG. 5: a result diagram of the Dendrobium nobile in increasing fresh weight and dendrobine content after co-cultured with endophytic fungus Phyllosticta fallopiae DNL19.

FIG. 6: a gene expression heat map of the Dendrobium nobile in increasing fresh weight and dendrobine content after co-cultured with endophytic fungus Phyllosticta fallopiae DNL19.

FIG. 7: a key gene expression diagram of the Dendrobium nobile in increasing fresh weight and dendrobine content after co-cultured with endophytic fungus Phyllosticta fallopiae DNL19.

### DESCRIPTION OF EMBODIMENTS

An endophytic fungus of the present disclosure is derived from separation and purification in leaves of an Orchidaceae plant, Dendrobium nobile. The present disclosure is further described in combination with specific embodiments and accompanying drawings followed.

### Example 1

The endophytic fungus was obtained by separation according to the following steps:

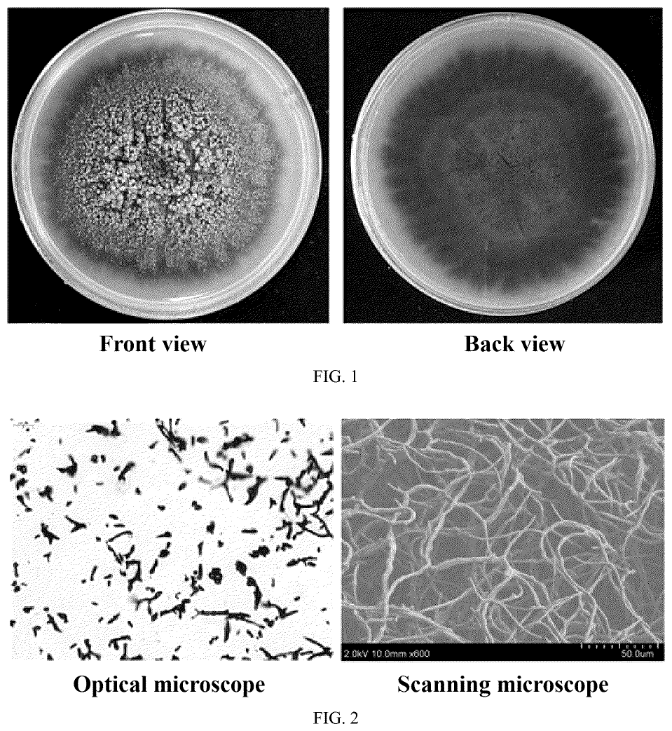

Root, stem and leaf samples of Dendrobium nobile were flushed with clear water for 5 min and then flushed with sterile water, then moisture on surfaces was absorbed to dryness using sterile filter paper, and next, surface disinfection was carried out on the samples using a three-step disinfection method (root: 75 vol % C₂H₅OH for 1 min, 2.5 wt % NaClO for 4 min, and 75 vol % C₂H₅OH for 1 min; stem: 75 vol % C₂H₅OH for 1 min, 2.5 wt % NaClO for 3 min, and 75 vol % C₂H₅OH for 1 min; and leaf: 75 vol % C₂H₅OH for 1 min, 2.5 wt % NaClO for 1 min, and 75 vol % C₂H₅OH for 30 s). After the three-step disinfection method, the samples were flushed with sterile water 5 times, and next, moisture on the surfaces of the samples was absorbed to dryness using sterile filter paper. The roots, stems and leaves of the Dendrobium nobile were sheared into tissue blocks using sterile scissors and forceps, with the stems and the roots 0.5 cm long and the leaves 0.5 cm×0.5 cm. Afterwards, traditional separated culture was carried out, 30 tissue blocks were randomly picked from each of the 3 different tissue parts, namely the roots, stems and leaves, and every 4-5 tissue blocks were placed in a flat plate of a PDA medium containing penicillin (50 mg/L) and placed in a 25° C. constant-temperature and constant-humidity incubator to be cultured. Growth situations of hyphae were observed regularly. Afterwards, tip hyphae were picked and transferred to a new PDA medium, such that a single fungal colony was cultured through separation and purification, and by continuously repeating this, a purified strain was obtained. The purified strain was divided into different morphotypes, and finally the endophytic fungus DNL19 of the present disclosure was obtained. As shown in FIG. 1, culture was carried out in darkness at 25° C. on a potato-glucose-agar medium (PDA), the growth of the strain was slow, and fungal colonies are in dark grey, with white circular granular protrusions in the middle circle and smooth off-white fungal colonies in the outer circle, see FIG. 1. Under an optical microscope, the fungal colonies were in black, purple with short and thin hyphae and many branches, and internal diaphragms of part of the hyphae were seen indistinctly. Under a scanning microscope, hyphae were circular as a whole, part of the hyphae were flat occasionally, walls were relatively smooth, and the hyphae were thin and had many branches, see FIG. 2. The strain was preserved at a low temperature of 4° C.

DNA of the purified endophytic fungus strain from the roots, stems and leaves of the Dendrobium nobile was extracted by adopting a DNAiso Reagent genome DNA purification kit. PCR amplification was carried out on primers by adopting ITS4 (SEQ ID NO. 2: 5′-TCCTCCGCTTTATTGATATGC-3′) and ITS5 (SEQ ID NO. 5′-GGAAGTAAAGTCGTAACAAGG-3′). A PCR system (50 μL) was composed of: template DNA, 2 μL; primer ITS4, 1 μL; primer ITS5, 1 μL; 2×TapPCR MasterMix (containing dye liquor), 25 μL; and ddH₂O, 21 μL. Cyclic conditions for a PCR: initial denaturation at 95° C. for 3 min; denaturation at 94° C. for 40 s; annealing at 52° C. for 50 s; extension at 72° C. for 1 min; 35 denaturation-annealing-extension cycles; and final extension at 72° C. for 10 min. Whether PCR products were qualified or not was detected via 1% agarose gel electrophoresis detection, and qualified PCR amplification products were sent to Shanghai Sangon Biotech CO., Ltd. for performing sequencing. A sequence is as shown in SEQ ID NO. 1.

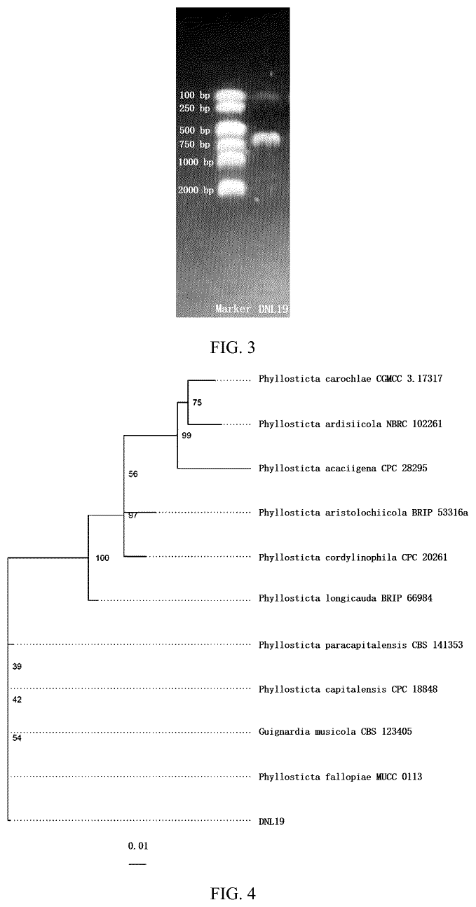

With the sequence obtained by PCR product sequencing as a target sequence, homologous sequences were searched in the GenBank database in NCBI, reference sequences which were the most similar to the morphotype sequence were downloaded, phylogenetic analysis was carried out using a neighbor-joining (NJ) method, a phylogenetic position of a to-be-identified strain was determined, and as shown in FIGS. 3 and 4, the homology with Phyllosticta fallopiae was 99.83%. Therefore, the endophytic fungus of the present disclosure is classified and named Phyllosticta fallopiae DNL19.

The endophytic fungus isolated from Dendrobium nobile has been deposited in the China General Microbiological Culture Collection Center on Apr. 27, 2023, and is classified and named Phyllosticta fallopiae DNL19, with a deposit number of CGMCC No. 40583.

### Example 2

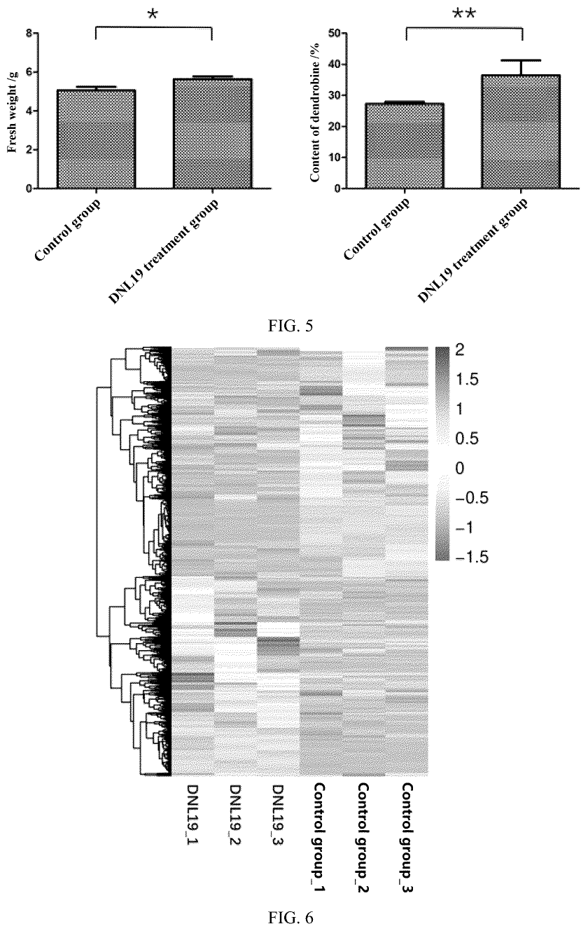

A DNL19 freezing tube was taken under a sterile condition, a certain number of hyphae were picked with an inoculating needle, and the hyphae were inoculated into a PDA medium for performing activation of culture and cultured to be mature at 25° C. under a 65% humidity condition. Fungus blocks at an edge of fungal colonies were punched with a 5 mm puncher; the obtained fungus blocks were placed in a ½ MS medium for co-culture with Dendrobium nobile tissue culture seedlings, and the fungus blocks were placed in the middle of the tissue culture seedlings. For a control group, an empty PDA medium was used, and fungus blocks were also inoculated in the middle of the tissue culture seedlings to be cultured. The tissue culture seedlings were placed in a greenhouse with a temperature of 25±1° C., an illumination/dark cycle of 12 h/12 h and a relative humidity of 60% to 70%, and culture time was 60 d. 30 repetitions were set in parallel. After culture was completed, 6 bottles of sterile Dendrobium nobile tissue culture seedlings were randomly selected to be used for measuring fresh weights and contents of dendrobine. The contents of the dendrobine were measured using a gas chromatographic method. A GC sample solution for being tested was prepared, and it was repeated 3 times for each sample. A Shimadzu SH-Rtx-1 capillary column (0.25 μm×0.25 mm×30 m) was used, N₂ was used as a carrier gas, a Shimadzu 2010 Plus gas chromatography-flame ionization detector (FID) was used, and reference was made to the Chinese Pharmacopoeia, 2020 version, for gas chromatography conditions. When a correction factor was measured, 1.0 μL of a dendrobine reference substance solution was precisely sucked using a micro-syringe, and was injected into a gas chromatograph, compositions of a total ion chromatogram were extracted through the flame ionization detector, sample injection was continuously repeated 3 times, a peak area of the chromatogram was recorded, and the correction factor F was calculated. A standard curve regression equation of the dendrobine was Y=794.74X−7191.50 (R²=0.990), and the linearity range was 20-200 μg/L. It was found according to the results that, the fresh weight of the Dendrobium nobile inoculated with the DNL19 was increased by 11.44% compared to the control group, the content of the dendrobine was increased by 33.80%, as shown in FIG. 5, and it indicated that inoculating with the DNL19 can remarkably promote the growth and dendrobine accumulation of Dendrobium nobile.

Dendrobium nobile tissue culture seedlings were taken from the DNL19 treatment group and the control group, total RNA was extracted using a TaKaRa MiniBEST Universal RNA Extraction Kit, 3 μL of an RNA sample was placed into 1.0% agarose gel and subjected to 120 V electrophoresis for 30 min, and a gel imager was used for observation; and 1 μL of an RNA sample was taken and diluted by adding an appropriate amount of ddH₂O, and OD values were measured under 260 nm and 280 nm. A transcriptome library was constructed after RNA was qualified in quality testing, paired-end sequencing was carried out using Illumina Hiseq2000/2500 after the library was qualified in quality testing, after quality control was performed on sequencing data, Trinity software was used for splicing the sequencing data, after being obtained by splicing, genes were aligned with sequences in 5 public databases (Swiss-Prot, NR, KEGG, KOG and Pfam), with a threshold e≤1e⁻¹⁰, function annotation was performed through sequence similarities, and a BLAST algorithm was used for sequence similarity alignment. A significance test of difference was performed on reads by adopting a negative binomial distribution test in a DEGseq software package, an expression amount of a unigene was estimated by adopting a basemean value, and differential genes related to growth of the Dendrobium nobile and metabolism of the dendrobine after DNL19 treatment were screened. The expression amounts of the differential genes are as shown in FIG. 6.

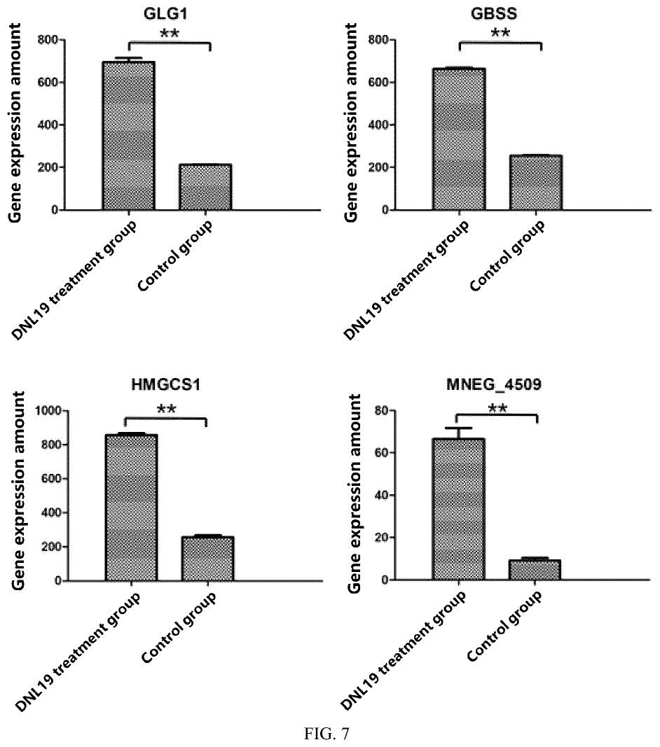

Further, the expression amounts of the differential genes were verified by adopting a reverse transcription system PrimerScript RT kit. A reaction has two steps: (1) a 10 μL reaction system: 5×gDNA Eraser Buffer 2 μL, gDNA Eraser 1 μL, RNA 2/c μL (c is an RNA concentration, μg/μL), supplementing to 10 μL with DEPC H₂O, 42° C., 2 min; (2) a 20 μL reaction system: a product from step (1) 10 μL, PrimeScript RT Enzyme Mix I 1 μL, RT Primer Mix 1 μL, 5×PrimerScript Buffer 2 (for Real Time) 4 μL, EASY Dilution 4 μL, 37° C., 15 min, 85° C. 5 s, and reverse transcription products diluted by 2 folds for standby use. BLAST alignment was performed on a CDS sequence of a target gene, by selecting an appropriate segment, primers were designed using Primer Premier 5.0 software, and primer sequences were as shown in Table 1, with β-actin as a reference gene. A Real-Time PCR system, a total volume of 10 μL, SYBR 5 μL, Primer-F 0.4 μL, Primer-R 0.4 μL, RNase free ddH₂O 3.7 μL, cDNA 0.5 μL. Reaction procedures: 95° C., 30 s; 95° C., 5 s, 60° C., 30 s, 72° C., 30 s, 39 cycles; and 65° C., 5 s, 95° C. 5 s. After being treated with the endophytic fungus DNL19, GLG1, GBSS, HMGCS1 and MNEG_4509 genes of the Dendrobium nobile obtained by screening had remarkably increased expression amounts, as shown in FIG. 7. It indicated that the endophytic fungus DNL19 increased the fresh weight and dendrobine content of Dendrobium nobile by up-regulating the expression key genes such as GLG1, GBSS, HMGCS1 and MNEG 4509.

TABLE 1
Primer Sequences

| Gene | Forward primers Primer-F | Reverse primers Primer-R |
|---|---|---|
| GLG1 | SEQ ID NO. 4: | SEQ ID NO. 5: |
| | AAGGGCGTATAGTTGAGT | AAGGGCGTATAGTTGAGT |
| GBSS | SEQ ID NO. 6: | SEQ ID NO. 7: |
| | GGCTTATCTTATGTGGCA | AAAGGCTGAACCGTATCT |
| HMGCS1 | SEQ ID NO. 8: | SEQ ID NO. 9: |
| | CGCTTGGAAGTTGGTAGT | GTGGGTCCCTCTGTATTT |
| MNEG_4509 | SEQ ID NO. 10: | SEQ ID NO. 11: |
| | TCATCAAGGAAGCAGGAG | AAAGGCGAAATCAAACAG |

The preferred examples of the present disclosure have been described in detail above, but the present disclosure is not limited to these examples. Those skilled in the art may make various equivalent modifications or replacements without departing from the creative spirit of the present disclosure, and these equivalent modifications or replacements are all included within the scope defined by the claims of the present application.

What is claimed is:

1. An endophytic fungus of Dendrobium nobile, wherein the endophytic fungus of Dendrobium nobile has been deposited the China General Microbiological Culture Collection Center on Apr. 27, 2023, with a category name of Phyllosticta fallopiae DNL19, and a deposit of CGMCC No. 40583.

2. A use of the endophytic fungus of Dendrobium nobile according to claim 1, wherein the use is a use of the endophytic fungus of Dendrobium nobile in improvement of a yield and quality of Dendrobium nobile.

3. The use according to claim 2, wherein the use is that: inoculating an endophytic fungus cake into a Dendrobium nobile growth medium for co-culture.

4. The use according to claim 3, wherein inoculating the endophytic fungus cake into the Dendrobium nobile growth medium for performing co-culture comprises:
(1) taking a strain of endophytic fungus of Dendrobium nobile, under a sterile condition, picking hyphae with an inoculating needle, inoculating the hyphae on a sterilized PDA medium, and carrying out dark culture for 7 d at 25° C. under a 65% humidity condition; and
(2) under a sterile condition, punching fungus blocks, namely the endophytic fungus cake, at an edge of fungal colonies with a sterilized puncher, inoculating the fungus cake into a ½ MS medium for co-culture with Dendrobium nobile tissue culture seedlings, placing the fungus block in the middle of the tissue culture seedlings, and placing the medium in a greenhouse to carry out co-culture with a culture temperature of 25±1° C., illumination/dark 12 h/12 h, and a relative humidity of 60% to 70%.

5. The use according to claim 4, wherein a formula of the PDA medium is: 200 g potato, 20 g glucose, 15 g agar, and 1000 mL distilled water, with a natural pH.

6. The use according to claim 4, wherein a formula of the ½ MS medium is: 1.9 g potassium nitrate, 1.65 g ammonium nitrate, 0.17 g potassium dihydrogen phosphate, 0.18 g magnesium sulfate, 0.33 g calcium chloride, 0.83 mg potassium iodide, 6.2 mg boric acid, 16.9 mg manganese sulfate, 8.6 mg zinc sulfate, 0.25 mg sodium molybdate, 0.025 mg copper sulfate, 0.1 g inositol, 0.025 mg cobalt chloride, 2.0 mg glycine, 37.26 mg EDTA sodium salt, 0.1 mg vitamin B1, 15.2 mg ferrous sulfate, 0.5 mg nicotinic acid, 0.5 mg vitamin B6, 15.0 g sucrose, 7.0 g agar powder, and 1000 ml distilled water, with a pH of 5.8-6.0.
